(19) United States
(12) Patent Application Publication
(10) Pub. No.: US 2002/0026167 A1
(43) Pub. Date: Feb. 28, 2002

(54) Feminine protective pad

(76) Inventor: Paula Sue Pompa, Albuquerque, NM (US)

Correspondence Address: Richard E. Backus, Law Offices of Richard E. Backus, Suite 490, 685 Market Street, San Francisco, CA, 94105, US

(21) Appl. No.: 09/931,622

(22) Filed: Aug. 17, 2001

Related U.S. Application Data
(60) Provisional application No. 60/226,945, filed on Aug. 22, 2000.

(51) Int. Cl. A61F 13/15

(52) U.S. Cl. 604/378; 604/385.01

(57) ABSTRACT

A feminine protective pad for use during a woman's menstrual cycle. The pad has top and bottom layers, each of which includes a liquid impervious garment-facing side and a liquid pervious body-facing side. A core of liquid-absorbent material is sandwiched between the two sides. The core is sufficiently hydrophilic to absorb menstrual liquids. The outer margins of the layers are releasably joined together so that a user can remove and discard the top layer when it becomes soiled. This enables the bottom layer to continue to be worn while obviating the need for the user to carry replacement pads in her purse, backpack or other container.

### CROSS-REFERENCE TO PRIOR APPLICATION

This application claims the benefit under 35 USC §119(e) of United States provisional application Serial No. 60/226,945 filed Aug. 22, 2000.

### BACKGROUND OF THE INVENTION

1. Field of the Invention

This invention relates in general to feminine protection during the menstrual cycle. In particular, it relates to protective pads for use by women during their menstrual cycles.

2. Description of the Related Art

One conventional means for feminine protection when a young girl first starts her menstrual cycle is to attach a single layer protective pad to her undergarments.

Another means is for a woman to insert a tampon. Along with a tampon, many women also use a single layer pad secured to her undergarments for extra protection. Some women also choose to use only single layer pads attached to their undergarments.

A common form of a absorbent layer for feminine protective pads comprises a liquid pervious topsheet, an absorbent core and a liquid impervious backsheet, such as a polyethylene film.

Conventional protective pads have mechanical fasteners made of adhesives that attach to the women's undergarments. Once soiled, they need to be replaced and thrown away. Such pads are replaced by detaching the soiled pad and then replacing it with a new pad. This requires the woman to carry a supply of the pads in a purse, backpack or the like from which it can be retrieved, opened and then secured to the undergarments.

The need has therefore been recognized for a feminine protective pad which obviates the foregoing and other limitations and disadvantages of prior art pads of this type.

Despite the various such pads in the prior art, there has heretofore not been provided a suitable and attractive solution to these problems.

### OBJECTS AND SUMMARY OF THE INVENTION

It is a general object of the present invention to provide a new and improved feminine protective pad.

Another object is provide a protective pad of the type described in which a soiled layer of the pad can be easily discarded while allowing the woman to continue to have protection without the need to retrieve and fasten another fresh layer in place of the soiled one.

### BRIEF DESCRIPTION OF THE DRAWINGS

FIG. 1 is a perspective view of a feminine protective pad of the invention showing two layers of the pad partially separated.

FIG. 2 is a side elevation view of the protective pad of FIG. 1.

FIG. 3 is an enlarged cross sectional view taken along the line 3-3 of FIG. 2.

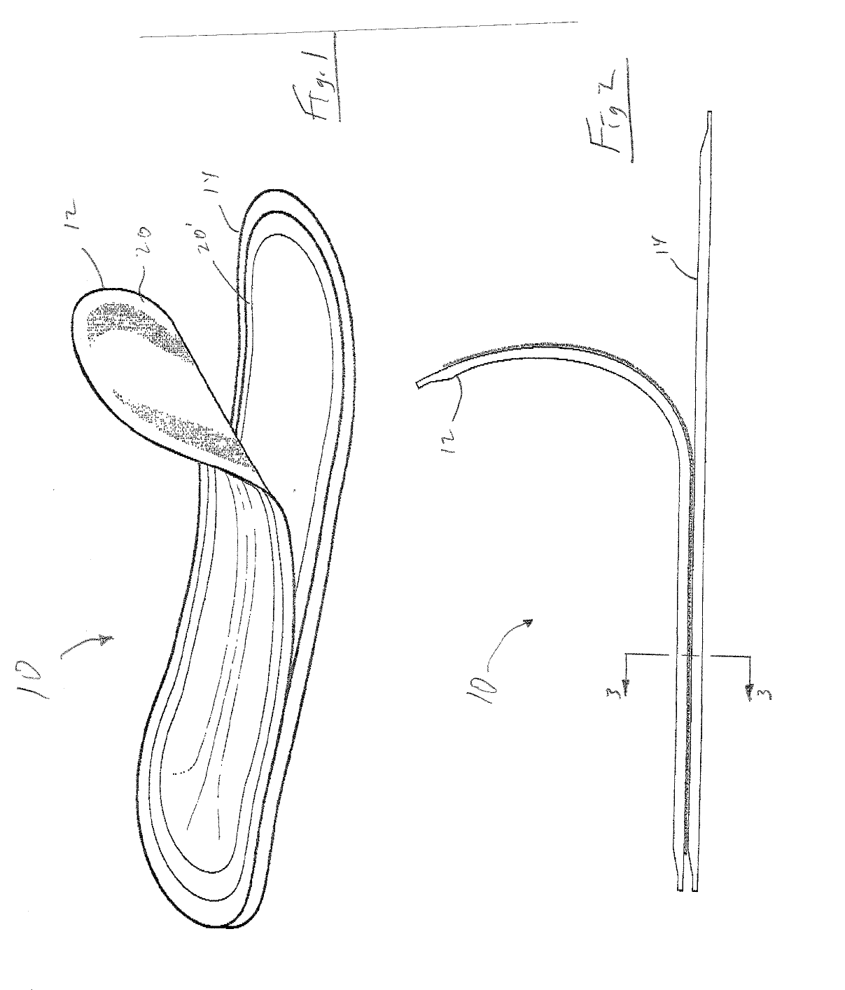

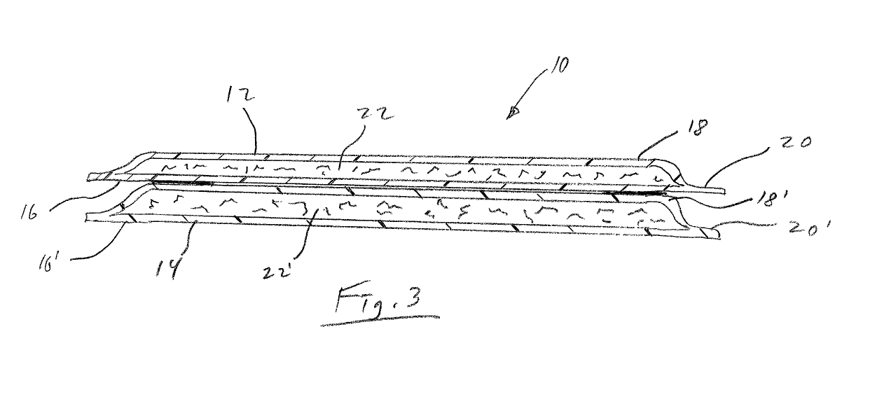

### DESCRIPTION OF THE PREFERRED EMBODIMENTS

In the drawings the figures show generally at 10 a feminine protective pad in accordance with one preferred embodiment. The pad 10 is comprised of multiple liquid-absorbing layers, of which two layers are shown: top layer 12 and bottom layer 14. The invention contemplates that the pad could be comprised of three or more such layers, as desired.

The layers 12 and 14 may be formed of suitable materials which are generally compressible, conformable, non-irritating to the wearers skin, and capable of absorbing and retaining liquids such as menstrual liquids. As best shown in FIG. 3, each layer 12 and 14 comprises, respectively, liquid impervious garment-facing sides 16, 16′, liquid pervious body-facing sides 18, 18′, outer perimeter margins 20, 20′ and absorbent cores 22, 22′.

Absorbent cores 22, 22′ are fabricated of a liquid absorbent material comprised of a suitable hydrophilic material. As used herein, the term “liquid absorbent material” refers to materials which wick, absorb and contain liquid body exudates, and, more specifically, refers to materials which can be placed against or in proximity with the body of the wearer to absorb and contain menstrual liquids as they are discharged from the body. The absorbent cores may be manufactured from a wide variety of liquid-absorbent materials commonly used in tampons and sanitary napkins, as well as other absorbent materials such as comminuted wood pulp which is generally referred to as airfelt. Examples of other suitable absorbent materials include craped cellulose wadding; meltblown polymers including coform; chemically stiffened, modified or cross-linked cellulosic fibers; tissues including tissue wraps and tissue laminates; absorbent foams; absorbent sponges; superabsorbent polymers; absorbent gelling materials; or any equivalent material or combinations of such materials.

The garment-facing sides 16, 16′ are comprised of a material which is sufficiently hydrophobic to inhibit the flow of menstrual liquids which would otherwise pass through and soil the user's garments. A layer of polyethylene film material is suitable for this purpose.

The body-facing sides 18, 18′ preferably are comprised of a suitable layer of synthetic polymer material which is sufficiently porous to enable menstrual liquids to pass through into the absorbent cores. The liquid absorbent material in the core could also be suitably selected so that the core's upper surface forms the liquid pervious body-facing side, thereby eliminating the need for a separate pervious layer.

The top and bottom layers 12 and 14 are shaped with generally flat, elongated configurations for a comfortable conforming fit with the user's genital anatomy. The layers are stacked together with their facing sides in juxtaposed relationship. The outer perimeter margins 20 and 20′ of the layers are releasably secured together by suitable means such as a thin film of weakly holding adhesive 24. This enables the user to manually peel the layers apart when the one adjacent the body becomes soiled.

When both layers are worn together, hydrophobic garment-facing side 16 of the top layer prevents menstrual liquid that is being absorbed by upper core 22 from passing down into core 22′ of the bottom layer.

After the top layer becomes soiled it can then be disposed of for enabling the user to continue wearing the bottom layer to absorb flow of additional menstrual liquids. This allows the user to comfortably wear multiple liquid-absorbing layers while enabling convenient removal of a soiled layer. A fresh layer is thereby exposed for use without the need for carrying replacement protective pads in a purse, backpack or the like.

In cases of heavy menstrual flow, several layers may need to be discarded in the foregoing manner. However, the pad 10 will still protect undergarments of the user from being soiled from such heavy flow.

What is claimed is:

1. A feminine protective pad for use during a woman's menstrual cycle comprising the combination of top and bottom layers, each layer comprising a garment-facing side which is sufficiently hydrophobic to inhibit the flow of menstrual liquids, a body-facing side, and a core of liquid-absorbent material positioned between the garment-facing and body-facing sides, the core being sufficiently hydrophilic to absorb menstrual liquids, the top layer being positioned with its garment-facing side in juxtaposed relationship with the body-facing side of the bottom layer.

2. A feminine protective pad as in claim 1 which further comprises a releasable structure which releasably holds the outer perimeter margins of the layers together.

3. A feminine protective pad as in claim 2 in which the releasable structure comprises a layer of adhesive between the outer perimeter margins.

4. A feminine protective pad as in claim 1 in which the garment-facing side is comprised of a layer of polyethylene material.

5. A feminine protective pad as in claim 1 in which the body-facing side is comprised of a layer of material which is sufficiently porous to enable passage of menstrual liquids.

6. A feminine protective pad as in claim 5 in which the material of the body-facing side is a porous synthetic polymer.
